(19) United States
(12) Patent Application Publication
(10) Pub. No.: US 2002/0187488 A1
(43) Pub. Date: Dec. 12, 2002

(54) Quantitative assay for nucleic acids

(76) Inventors: Chao Lin, Brookline, MA (US); Anne Dak-Yee Kwong, Cambridge, MA (US)

Correspondence Address: Tina Powers, VERTEX PHARMACEUTICALS INC., 130 Waverly Street, Cambridge, MA, 02139-4242, US

(21) Appl. No.: 10/060,738

(22) Filed: Jan. 30, 2002

Related U.S. Application Data
(60) Provisional application No. 60/265,143, filed on Jan. 30, 2001.

(51) Int. Cl. C12Q 1/68; C12P 19/34

(52) U.S. Cl. 435/6; 435/91.2

(57) ABSTRACT

The present invention provides a method of accurately assaying the amount of nucleic acids in a biological source. According to another embodiment, the present invention provides a method of accurately assaying HCV in a biological source. The present invention also provides a method of simultaneously screening the effect of a plurality of compounds on the replication of a whole or part of a genome of a biological source. The present invention provides a method of simultaneously screening the effect of a plurality of compounds on the replication of the whole or part of the HCV genome in a biological source.

### CROSS REFERENCE TO RELATED APPLICATIONS

This application claims priority to co-pending U.S. provisional application No. 60/265,143, which was filed Jan. 30, 2001.

### BACKGROUND OF THE INVENTION

The detection and quantification of nucleic acids is useful in assaying its biological source. For example, Hepatitis C Virus (HCV) is a positive stranded RNA virus that has been shown to be the etiological agent responsible for the vast majority of transfusion and community associated non-A non-B viral hepatitis cases. It is considered an important cause of chronic hepatitis, cirrhosis, and end stage liver disease. HCV assays that are rapid and reproducible are crucial for monitoring HCV therapies. Thus, highly specific and sensitive assays that detect ad quantify HCV RNA can be used for this purpose.

One method known in the prior are for assaying such a biological material involves amplification procedures based on a branched-DNA method, in which a signal previously hybridized with the template sequence is amplified. But there is no internal control for the bDNA assay to monitor the effects of any inhibitors. Moreover, the sensitivity of the assay is limited by the fact that detection of fewer than 200,000 copies per ml of sample is precluded.

Another method involves reverse-transcription-PCR (“RT-PCR”), in which a viral genome sequence is directly amplified. Quantitative PCR methods known in the art typically measure the amount of amplified product at the end of the amplification reaction. RT-PCR based assays, although sensitive, display poor reproducibility and are time consuming. For this reason, this technique is not suited for high throughput screening of a plurality of compounds. Moreover, inhibitors present in body fluids may inhibit the reaction, resulting in false or inaccurate determinations of low copy numbers. And, because the end product of the reaction is quantified, small errors in the amplification step can contribute to false results.

Another commercially available technique, namely, the Amplicor technology, does employ an internal control. But, the internal control therein is not amplified in the same tube as the sample being studied. Moreover, the internal control is unrelated to the HCV RNA moiety. As a result, any error in the extraction or amplification of the HCV virus is left unmonitored and/or uncorrected.

Thus, the above methods known in the art have one or more of the following drawbacks: (i) a lack of a proper internal control for evaluating the efficiency of viral RNA extraction and accuracy of the RT-PCR reaction (ii) a high level of variability, (iii) a limited range of detection due to endpoint rather than real-time detection, (iv) a lack of sensitivity, and (v) a low throughput assay, not suitable for quick screening of a plurality of compounds.

There is a need for a method of accurately assaying the amount of nucleic acids in a biological source.

There is also a need for a method of accurately assaying HCV in a biological source.

There is a need for a method of simultaneously screening the effect of a plurality of compounds on the replication of a whole or part of a genome in a biological source.

There is also a need for a method of simultaneously screening the effect of a plurality of compounds on the replication of the whole or part of the HCV genome.

### SUMMARY OF THE INVENTION

It is an object of the present invention to provide a method of accurately assaying the amount of nucleic acids in a biological source.

It is another object of the present invention to provide a method of accurately assaying HCV in a biological source.

It yet another object of the present invention to provide a method of simultaneously screening the effect of a plurality of compounds on the replication of a whole or part of a genome of a biological source.

It is yet another object of the present invention to provide a method of simultaneously screening the effect of a plurality of compounds on the replication of the whole or part of the HCV genome in a biological source.

### DETAILED DESCRIPTION OF THE INVENTION

According to one embodiment, the present invention provides a method of quantifying a first nucleic acid in a first biological source, comprising the steps of:

(a) combining said first biological source containing said first nucleic acid with a known amount of a second biological source containing a second nucleic acid;

(b) extracting from said combination said first nucleic acid and said second nucleic acid to form a combined nucleic acid extract;

(c) adding to said combined nucleic acid extract a first detectable probe which is specific for said first nucleic acid and a second detectable probe which is specific for said second nucleic acid;

(d) amplifying said combined nucleic acid extract by PCR means with a first set of primers which is specific for said first nucleic acid and a second set of primers which is specific for said second nucleic acid;

(e) quantifying at various PCR cycles during said amplification a detectable signal released independently from said first detectable probe and said second detectable probe;

(f) extrapolating the results of step (e) to calculate the amount of said first nucleic acid in said first biological source and the amount of said second nucleic acid in said second biological source; and

(g) evaluating accuracy of said calculated amount of said first nucleic acid determined in step (f) by comparing said calculated amount of said second nucleic acid in step (f) with said known amount of said second nucleic acid used in step (a).

According to a another embodiment, the above method comprises the additional step of adjusting said calculated amount of said first nucleic acid determined in step (f) by a factor determined by comparing said calculated amount of said second nucleic acid in step (f) with said known amount of said second nucleic acid used in step (a).

In the method of the present invention, the first biological source is selected from cell-associated virus, including virus particles, sub-particles or free nucleic acid. Alternatively, the first biological source can be a cell-free virus, including virus particles, sub-particles or free nucleic acid in a suitable media such as serum or plasma media.

In a preferred embodiment, the first biological source is a cell-associated virus.

The first nucleic acid in the methods of the present invention is selected from viral DNA or viral RNA. In a preferred embodiment, the viral DNA or viral RNA is present in a cell-associated virus. According to another preferred embodiment, the viral DNA or viral RNA is present in a cell-free virus.

The second biological source in the methods of the present invention is selected from cell-associated virus, including virus particle, sub-particle or free nucleic acid. Alternatively, the second biological source can be a cell-free virus, including serum, plasma or any other media containing virus particle, sub-particle or free nucleic acid.

The second biological source is selected such that it is closely related to the first biological source. For the purposes of the present invention, the phrase “closely related” means similar biological characteristics of the first and second biological sources, such as, e.g., similar nucleic acids.

The presence of a related second biological source in the same well as the first biological source is key to the present invention. The second biological source serves as an internal control for the quantification of the first nucleic acid. This internal control feature allows for the monitoring and correction of random fluctuations and assay variability. These fluctuations and variability can result from specimen handling and storage, the presence of PCR inhibitors in body fluid samples, variability among lots of biochemical reagents, different methodologies, and random variations both in preparations and testers. Because the second biological source is closely related to the first biological source, its use as an internal control diminishes or even eliminates false-negative results and provides a more accurate picture of the level of the first nucleic acid.

The amplification step in the methods of the present invention is typically conducted using PCR means. One of skill in the art will be well aware of PCR means and attendant strategies useful in the methods of the present invention. See, e.g., “PCR Strategies”, Ed. Michael A. Innis, David H. Gelfand and John J. Sninsky, 1995, Academic Press.

In a preferred embodiment, the methods of the present invention use PCR or RT-PCR to amplify the combined nucleic acid extract. According to a more preferred embodiment, the methods of the present invention use RT-PCR to amplify the combined nucleic acid extract.

In the amplification step of the methods of the present invention, two sets of primers are used, a first set of primers specific for the first nucleic acid, and a second set of primers specific for the second nucleic acid.

Extraction means suitable for the present invention include any suitable DNA or RNA extraction techniques. Preferred extraction means include matrix-based single-well spin or vacuum column method, multiple-well extraction plate method or solution based-extraction methods. One of skill in the art would be well aware of commercially available systems such as QIAamp, RNeasy, or DNeasy Spin method columns, QIAamp, RNeasy, or DNeasy 96 well plates, Boom method (Chaotropic agent/glassbeads), Triazol, etc.

In step (b) of the method of the present invention, the nucleic acids of the first biological source and the nucleic acids of the second biological source are simultaneously extracted to produce a combined nucleic acid extract. The simultaneous extraction of nucleic acids is advantageous because the extraction efficiency affects the first and the second nucleic acid similarly. Thus, any random variation in the extraction process can be accounted for by the effect of the variation on the extraction of the second nucleic acid. Moreover, when the second biological source is closely related to the first biological source, the effect of such random variations on the first and second nucleic acid are likely to be very similar. As a result, the integrity of the second biological source as an internal control is enhanced.

In the methods of the present invention, two detectable probes are utilized to detect and quantify the first nucleic acid and the second nucleic acid. The two detectable probes are selected such that each is specific to one of the two nucleic acids. Thus, the first detectable probe is specific to the first nucleic acid, and not to the second nucleic acid. Similarly, the second detectable probe is specific to the second nucleic acid, and not to the first nucleic acid. Another criterion in the selection of the two detectable probes is that each should not interfere in the detection and quantification of the other. One of skill in the art would be well aware of detectable probes suitable for the present invention.

The property detected and quantified depends on the identity of the detectable probe selected. Examples of such properties include fluorescence, phosphorescence, color, etc.

In a preferred embodiment of the present invention, two different dual-labeled fluorogenic probes are used, each specific for one but not the other of the first nucleic acid and the second nucleic acid. In a more preferred embodiment, each fluorogenic probe typically has a reporter dye at the 5′-end and a quencher dye at the 3′ end. The two different fluorogenic probes are selected such that they give distinct fluorescence peaks that may be detected without cross-interference between the two peaks. For example, the 5′ end of the first detectable probe can be labeled with a reporter dye such as 6-carboxy-fluroscene (“6-FAM”), and the 5′ end of the second detectable probe can be labeled with a reporter dye such as VIC. The 3′ end of both detectable probes can be labeled with a quencher dye such as 6-carboxymethyl-rhodamine (“6-TAMRA”). Thus, when bound to the first nucleic acid and the second nucleic acid, the proximity of the reporter dye at the 5′ end to the quencher dye at the 3′ end of the probe results in a suppression of the fluorescence. During amplification, when the Tth polymerase moves along the nucleic acid sequence, the quencher is removed from the probe by the action of the 5′-3′ exo, thereby degrading the fluoregenic probe. This results in a fluorescence emission, which is recorded as a function of the amplification cycle. Thus, monitoring the fluorescence emission provides a basis for measuring real time amplification kinetics.

According to another embodiment, the present invention provides for quantifying a first nucleic acid in HCV, comprising the steps of:

(a) combining said HCV with a known amount of Bovine Viral Diarrhea Virus (“BVDV”), wherein said BVDV contains a second nucleic acid;

(b) extracting from said combination said first nucleic acid and said second nucleic acid to form a combined nucleic acid extract;

(c) adding to said combined nucleic acid extract a first detectable probe which is specific for said first nucleic acid and a second detectable probe which is specific for said second nucleic acid;

(d) amplifying said combined nucleic acid extract by PCR means;

(e) quantifying at various cycles during said amplification a detectable signal released independently from said first detectable probe and said second detectable probe;

(f) extrapolating the results of step (e) to calculate the amount of said first nucleic acid in said HCV and the amount of said second nucleic acid in BVDV; and

(h) evaluating accuracy of said calculated amount of said first nucleic acid determined in step (f) by comparing said calculated amount of said second nucleic acid in step (f) with said known amount of said second nucleic acid used in step (a).

According to another embodiment, the above method comprises the additional step of adjusting said calculated amount of said first nucleic acid determined in step (f) by a factor determined by comparing said calculated amount of said second nucleic acid in step (f) with said known amount of said second nucleic acid used in step (a).

According to another embodiment, the present invention provides a method of determining the effect of a compound on the replication of a first nucleic acid of a first biological source, comprising the steps of:

(a) combining said compound with a medium containing a known amount of said first biological source to produce a first combination, wherein said medium is suitable for replication of said first nucleic acid;

(b) after a time period combining said first combination with a second biological source containing a second nucleic acid to produce a second combination;

(c) extracting from said second combination said first nucleic acid and said second nucleic acid to form a combined nucleic acid extract;

(d) adding to said combined nucleic acid extract a first detectable probe which is specific for said first nucleic acid and a second detectable probe which is specific for said second nucleic acid;

(e) amplifying said combined nucleic acid extract by PCR means;

(f) quantifying at various PCR cycles during said amplification a detectable signal released independently from said first detectable probe and said second detectable probe;

(g) extrapolating the results of step (f) to calculate the amount of said first nucleic acid and said second nucleic acid in said second combination;

(h) determining the effect of said compound on the replication of said first nucleic acid by comparing said amount of said first nucleic acid determined in step (g) or (h) in the presence of said amount of said compound versus that in the absence of said compound

According to another embodiment, the present invention provides a method of simultaneously screening a plurality of compounds for their effect on the replication of a whole or part of a genome of a first biological source, comprising the steps of:

(a) placing in one or a plurality of wells said whole or part of a genome of said first biological and a medium suitable for replication of said genome;

(b) adding to each said well one or more of said compounds;

(c) adding to each said well a known amount of a second biological source as an internal control;

(d) using extraction means to extract together from each said well a first nucleic acid and a second nucleic acid to produce a combined nucleic acid extract from each well;

(e) amplifying and quantifying during the amplification process said first nucleic acid and said second nucleic acid in each well;

(f) determining the effect of each of said compounds on the replication of said whole or part of a genome of a first biological source using the results from step (e).

The compound selected is such that it has no effect on the concentration of the second nucleic acid. Alternatively, the second virus is selected such that the concentration of its nucleic acid is not affected by the compound selected.

Preferably, the compounds selected for the above method are potential inhibitors of the replication of the whole or part of the genome of the first biological source.

The term ‘medium’, as used in the present invention, refers to the culture present in each well suitable for the replication of the whole or part of the genome of the first virus.

The term ‘whole or part of a genome’ refers to DNA or RNA sequences or parts thereof sought to be replicated.

The steps of extracting, amplifying and quantifying the first nucleic acid and the second nucleic acid are as described above.

In step (f) of the above method, the quantified amount of the nucleic acid of the first biological source (from step (e)), is used to determine whether the compound, added to the first virus in step (a), has affected the replication of the whole or part of the genome of the first virus. For example, if a compound has an inhibitory effect on the replication of the first biological source, such inhibition will lead to a lower value for the quantified amount of the first nucleic acid in step (e).

According to a preferred embodiment, the above method is used to simultaneously screen the effect of a plurality of compounds on the replication of a whole or part of a genome of HCV.

According to a more preferred embodiment, the above method is used to simultaneously screen the effect of a plurality of compounds on the replication of a whole or part of a genome of HCV, wherein BVDV is used as the internal control.

In order that this invention be more fully understood, the following examples are set forth. These examples are for the purpose of illustration only and are not to be construed as limiting the scope of the invention in any way.

### EXAMPLE 1

The method of the present invention is exemplified using HCV as the first virus and BVDV as the second virus.

Primers and Probe

The 5′ UTR sequences of 15 representative, HCV genotype 1 strains from Genbank were aligned using the DNA STAR program. Primers and probe were designed based upon most conserved regions. The probe was constructed based upon the following additional criteria: a) the melting temperature of the probe was 8° C. to 10° C. higher than that of the primers; b) no G's were present at the 5′ end; c) there is not a stretch of more than 4 G's; d) the probe does not form internal structures with high melting temperatures or form a duplex with itself or with any of the primers. The entire PCR region was about 150 base pairs in length.

The primers and probe for the 5′ UTR of BVDV were designed based on the same set of criteria. In addition, care was taken to ensure that the primers or probe of HCV has the least amount of homology to those of BVDV. The primers and probe for HCV genotype 1 are: 5′-CCATGAATCACTCCCCTGTG-3′ (forward primer), 5′-CCGGTCGTCCTGGCAATTC-3′ (reverse primer), and the HCV probe, 5′-6-FAM CCTGGAGGCTGCACGACACTCA-TAMRA-3′. The primers and probe for BVDV comprised the forward primer, 5′-CAGGGTAGTCGTCAGTGGTTCG-3′, the reverse primer, 5′-GGCCTCTGCAGCACCCTATC-3′, and the probe, 5′-VIC CCCTCGTCCACGTGGCATCTCGA-TAMRA-3′. All primers and probes were obtained from Oligo, Etc, except for the BVDV probe (PE Applied Biosystems).

Preparation of Viral and Standard RNA

A 215 base pair cDNA fragment of the highly conserved 5′ UTR of HCV genotype was selected as the template for generation of HCV (+) strand RNA standard.

MDBK cells were infected with BVDV NADL strain. The progeny BVDV was harvested from the mixture of cell lysate and extracellular supernatant and the viral RNA was extracted using the QIAamp spin column methodology (QIAGEN) as outlined by the manufacturer.

HCV positive sera were obtained from a commercial vendor (ProMedx) and the HCV concentration was determined using the Chiron bDNA assay. HCV negative human sera were obtained from Sigma (catalog #S-7023). 140 μl of human sera was spiked with a fixed amount of BVDV and extracted using QIAamp spin columns. 20 μl of RNA extracts were taken for each PCR reaction.

Taqman Real Time RT-PCR Assay

The RT and the PCR reactions were carried in the same wells of a 96 well plate optical tray with caps (PE Applied Biosystems, Foster City, Calif.). For the singleplex Taqman assay with only one viral RNA, 10 or 20 μl of viral RNA or RNA standard was amplied in a 50 μl RT-PCR reaction with 1XTaqman EZ buffer (PE Applied Biosystems), 3 mM Manganese acetate, 300 μM each of dATP, dCTP, dGTP, and dUTP, 200 nM 6-FAM-labeled HCV probe or VIC-labeled BVDV probe, 200 nM HCV or BVDV primers, 6 units Tth polymerase (Epicentre), and 4.0% enhancer (Epicenter). The Taqman RT-PCR assay was run for 25 min at 60° C. (RT), 5 min at 95° C., and followed by 45 cycles of two-step PCR reaction (60° C. for 1 min and 95° C. for 15 sec). For the multiplex Taqman assay, the amount of HCV and BVDV primers was optimized using a matrix mixture of various concentration of both sets of primers. The final assay condition includes 200 nM of both 6-FAM-labeled HCV probe and VIC-labeled BVDV probe, 400 nM of both HCV primers, and 45 nM of both BVDV primers.

Table 1 compares a singleplex assay with a typical multiplex assay run using our system. In this case, 50, 100, 1000, 10⁴, and 10⁶ copies of HCV RNA standard were analyzed with (multiplex) or without (singleplex) BVDV internal control RNA. The standard curve for HCV was established with a set of HCV RNA standard without BVDV internal control RNA. A correlation coefficient of more than 0.98 was observed in the range of 50 to 10⁷ copies of HCV RNA in the standard curve. As shown in table 1, there is little difference of the HCV Ct values or RNA copy numbers between the multiplex and singleplex assays. The Ct values of BVDV RNA internal control range from 20.32 to 21.28, with an average of 20.77. These data indicate that there is no interference from BVDV internal control RNA on the quantification of HCV RNA level in our multiplex assay. Both types of nucleic acid were measured accurately at the same time and in one RT-PCR tube. Up to 10⁷ copies of HCV RNA was measured accurately in this multiplex assay. These results indicate that the dynamic range of this multiplex assay is from 50 to 10⁷ copies of HCV RNA. This assay can be modified to measure more than 10⁷ copies of HCV RNA if the amount of BVDV internal control RNA is increased.

Table 2 displays the reproducibility of this multiplex using the in vitro transcribed RNA. 50, 100, 1000, 10⁴, and 10⁶ copies of HCV RNA was tested with BVDV internal control RNA in quadruplicate. The same assay was run twice over two days. Similar Ct values or the copy number of HCV RNA were observed for both days. The % CV of the intra- and inter-assay was at similarly low level for either Ct values or the copy number of HCV RNA. These results clearly demonstrate that this multiplex assay can be used to measure HCV RNA level with excellent accuracy and reproducibility, and with a great dynamic range.

In addition, several HCV positive patient sera samples were obtained from commercial source and tested in our multiplex assay. The HCV viral load in these sera has been measured by the vendor using the bDNA assay. The HCV serum samples were extracted along with a fixed amount of BVDV using the QIAamp spin column technique.

Table 3 shows the results of the multiplex assay for a representative serum sample (#864) from HCV genotype la. As may be seen from Table 3 there is an excellent correlation among the 10-fold serial dilution of the same serum sample, up to 1:10,000 dilution. The dynamic range in this is almost 5 log, from 31 to 1.14×10⁵ (undiluted) copies of HCV RNA. The HCV RNA level determined using our multiplex assay was from 2.66×10⁶ to 7.23×10⁶, which is close to the level (7.4×10⁶) determined by the commercial bDNA method.

In addition, two more HCV patient serum, one of type 1a and the other type 1b, were extracted with BVDV internal control and tested in our multiplex assay system. As can be observed in Table 4, two different dilutions of either serum resulted in the similar final titer of HCV RNA for the same serum. These results indicate that the multiplex assay can be used to quantify both HCV types 1a and 1b serum.

### EXAMPLE 2

A stable Huh7 cell line in which HCV RNA replication was established using a selectable marker. This cell line was used to test HCV inhibitors using our multiplex assay system. A DMSO stock of one of the HCV inhibitors was serially diluted into tissue culture media and incubated with a fixed number of the HCV replicon Huh7 cells in 96-well culture plate. The total cellular RNA in each culture well was extracted with RNeasy-96 extraction plate, along with a known amount of BVDV virus as internal control. The combined RNA extract (in 96-well format) was subject to the multiplex assay (for both HCV and BVDV).

Table 5 shows the results of such a typical experiment. For each sample, both HCV and BVDV Ct values were simultaneously determined, and the HCV RNA level was calculated using the HCV RNA standard curve shown in column 12. Wells H4 and H9 were shadow-colored, indicating failure or poor efficiency during extraction and/or RT-PCR since the BVDV signal in these two wells is significantly lower than that in other wells.

Table 6 shows the percentage of inhibition at various concentration of this HCV inhibitor on the HCV RNA level of the Huh7 stable cell line. An IC50 of 0.226 uM was calculated for this HCV inhibitor in this experiment. Several repeated experiments with the same HCV inhibitor resulted in IC50 values of 0.239, 0.345, 0.150, and 0.419 uM. These results demonstrate that the whole assay system, including the HCV replicon Huh7 stable cell line, 96-well culture with the potential HCV inhibitors, 96-well extraction of nucleic acid, and 96-well multiplex Taqman detection with an internal control, generated accurate, consistent, and reproducible results.

TABLE 1
Multiplex vs Singleplex Taqman Assay of HCV RNA Standard

| Input HCV | 50 | | 100 | | 1000 | | 10⁴ | | 10⁶ | |
|---|---|---|---|---|---|---|---|---|---|---|
| RNA (copy) | | RNA | | RNA | | RNA | | RNA | | RNA |
| HCV | Ct | copy | Ct | copy | Ct | copy | Ct | copy | Ct | copy |

Singleplex without BVDV
HCV RNA

| | | | | | | | | | | |
|---|---|---|---|---|---|---|---|---|---|---|
| average | 35.98 | 51 | 34.64 | 127 | 31.64 | 947 | 28.33 | 8.25 × 10³ | 21.21 | 9.31 × 10⁵ |
| % CV | 0.5% | 11.9% | 1.1% | 23.5% | 0.9% | 22.1% | 0.7% | 13.0% | 0.8% | 10.8% |

Multiplex with BVDV
HCV RNA

| | | | | | | | | | | |
|---|---|---|---|---|---|---|---|---|---|---|
| average | 36.19 | 45 | 34.60 | 133 | 31.67 | 901 | 28.35 | 8.11 × 10³ | 20.94 | 1.12 × 10⁶ |
| % CV | 1.0% | 24.6% | 1.5% | 33.2% | 1.0% | 19.5% | 0.4% | 8.1% | 1.5% | 22.2% |

BVDV RNA

| | | | | | |
|---|---|---|---|---|---|
| Average | 20.33 | 20.83 | 21.28 | 20.32 | 21.09 |
| % CV | 3.3% | 2.8% | 0.3% | 3.1% | 6.5% |

TABLE 2
Reproducibility of Multiplex Assay with HCV RNA standard

| Input HCV | 50 | | 100 | | 1000 | | 10⁴ | | 10⁶ | |
|---|---|---|---|---|---|---|---|---|---|---|
| RNA (copy) | | RNA | | RNA | | RNA | | RNA | | RNA |
| HCV | Ct | copy | Ct | copy | Ct | copy | Ct | copy | Ct | copy |

Intra-assay, day 1

| | | | | | | | | | | |
|---|---|---|---|---|---|---|---|---|---|---|
| Average | 36.69 | 34 | 34.44 | 145 | 31.05 | 1296 | 28.21 | 8.52 × 10³ | 20.60 | 1.05 × 10⁶ |
| % C | 0.83% | 20.24% | 0.77% | 16.09% | 1.22% | 25.91% | 2.81% | 40.95% | 1.48% | 19.40% |

Intra-assay, day 2

| | | | | | | | | | | |
|---|---|---|---|---|---|---|---|---|---|---|
| Average | 36.19 | 45 | 34.60 | 133 | 31.67 | 901 | 28.35 | 8.11 × 10³ | 20.94 | 1.12 × 10⁶ |
| % CV | 1.03% | 24.56% | 1.49% | 33.20% | 0.99% | 19.48% | 0.44% | 8.14% | 1.53% | 22.24% |

Inter-assay

| | | | | | | | | | | |
|---|---|---|---|---|---|---|---|---|---|---|
| Average | 36.40 | 41 | 34.52 | 139 | 31.30 | 1127 | 28.31 | 8.32 × 10³ | 20.72 | 1.08 × 10⁶ |
| % CV | 1.14% | 26.15% | 1.21% | 23.97% | 1.62% | 29.58% | 2.00% | 28.08% | 1.76% | 19.19% |

TABLE 3
Determination of viral load of HCV patient sera sample #864

| Patient | | | | Copy/ml | Copy/ml |
|---|---|---|---|---|---|
| sera | Dilution | Ct value | Copy/assay | (diluted) | (undiluted) |
| #864 | Neat | 24.41 ± | 1.14 × 10⁵ ± | 2.66 × 10⁶ ± | 2.66 × 10⁶ ± |
| (1a) | | 0.25 | 1.84 × 10⁴ | 4.29 × 10⁵ | 4.29 × 10⁵ |
| | 1:10 | 27.74 ± | 1.25 × 10⁴ ± | 2.91 × 10⁵ ± | 2.91 × 10⁶ ± |
| | | 0.06 | 495 | 1.15 × 10⁴ | 1.15 × 10⁵ |
| | 1:100 | 31.15 ± | 1.30 × 10³ ± | 3.02 × 10⁴ ± | 3.02 × 10⁶ ± |
| | | 0.08 | 77.8 | 1.81 × 10³ | 1.81 × 10⁵ |
| | 1:1000 | 34.47 ± | 143 ± 10.6 | 3.33 × 10³ ± | 3.33 × 10⁶ ± |
| | | 0.11 | | 247 | 2.47 × 10⁵ |
| | 1:10000 | 36.76 ± | 31.0 ± 1.41 | 723 ± 33.0 | 7.23 × 10⁶ ± |
| | | 0.07 | | | 3.30 × 10⁵ |
| | 1:100000 | 41.81 ± | 0 | 0 | 0 |
| | | 1.22 | | | |

TABLE 4
Determination of viral load of HCV patient sera samples

| Patient | Dilu- | | | Copy/ml | Copy/ml |
|---|---|---|---|---|---|
| sera | tion | Ct value | Copy/assay | (diluted) | (undiluted) |
| #898 | 1:10 | 25.49 ± 0.41 | 2.9 × 10⁴ ± | 6.7 × 10⁵ ± | 6.7 × 10⁶ ± |
| (1a) | | | 7.2 × 10³ | 1.7 × 10⁵ | 1.7 × 10⁶ |
| | 1:100 | 28.82 ± 0.35 | 3.0 × 10³ ± | 6.9 × 10⁴ ± | 6.9 × 10⁶ ± |
| | | | 6.6 × 10² | 1.5 × 10⁴ | 1.5 × 10⁶ |
| #865 | 1:10 | 27.61 ± 0.26 | 6.7 × 10³ ± | 1.6 × 10⁵ ± | 1.6 × 10⁶ ± |
| (1b) | | | 1.1 × 10³ | 2.5 × 10⁴ | 2.5 × 10⁵ |
| | 1:100 | 30.36 ± 0.65 | 1.1 × 10³ ± | 2.6 × 10⁴ ± | 2.6 × 10⁶ ± |
| | | | 4.6 × 10² | 1.1 × 10⁴ | 1.1 × 10⁶ |

TABLE 6
Inhibition of HCV RNA replication by a HCV inhibitor on a HCV replicon stable cell line

| HCV inhibitor | HCV RNA copy number | | | | | | | | % of |
|---|---|---|---|---|---|---|---|---|---|
| (uM) | #1 | #2 | #3 | #4 | #5 | average | SD | % CV | inhibition |
| 0 | 2.06E+07 | 2.23E+07 | 2.31E+07 | 1.98E+07 | 1.51E+07 | 1.76E+07 | 4.84E+06 | 27.48% | 0.00% |
| | 1.13E+07 | 9.65E+06 | 2.07E+07 | | 1.60E+07 | | | | |
| 0.01 | 1.09E+07 | 1.10E+07 | 1.83E+07 | 1.63E+07 | 1.86E+07 | 1.50E+07 | 3.80E+06 | 25.31% | 14.80% |
| 0.03 | 1.33E+07 | 1.83E+07 | 1.65E+07 | 1.71E+07 | 1.53E+07 | 1.61E+07 | 1.92E+06 | 11.93% | 8.65% |
| 0.1 | 1.06E+07 | 1.02E+07 | 1.81E+07 | 1.44E+07 | 2.28E+07 | 1.52E+07 | 5.29E+06 | 34.72% | 13.52% |
| 0.3 | 4.33E+06 | 6.90E+06 | 6.19E+06 | 3.40E+06 | 2.66E+06 | 4.70E+06 | 1.81E+06 | 38.46% | 73.34% |
| 1 | 8.83E+05 | 1.35E+06 | 1.33E+06 | 1.13E+06 | 1.41E+06 | 1.22E+06 | 2.16E+05 | 17.68% | 93.08% |
| 3 | 2.38E+05 | 2.86E+05 | 2.33E+05 | 2.43E+05 | 3.39E+05 | 2.68E+05 | 4.51E+04 | 16.84% | 98.48% |

IC50 = 0.226 uM

TABLE 5
Test of a potential HCV inhibitor on HCV RNA replication of a HCV replicon stable cell line using 96-well
RNA extraction and Multiplex Taqman

| | 1 | | 2 | | 3 | | 4 | | 5 | | 6 | |
|---|---|---|---|---|---|---|---|---|---|---|---|---|
| | HCV | BVDV | HCV | BVDV | HCV | BVDV | HCV | BVDV | HCV | BVDV | HCV | BVDV |
| A | 21.1 | 21.5 | 21.1 | 21.3 | 20.4 | 20.4 | 20.5 | 21.1 | 20.3 | 20.5 | 19.8 | 20.4 |
| | ####### | | ####### | | ####### | | ####### | | ####### | | ####### | |
| B | 20.8 | 21.7 | 20.3 | 21.3 | 20.5 | 21.1 | 20.5 | 20.3 | 20.6 | 20.7 | 20.3 | 20.3 |
| | ####### | | ####### | | ####### | | ####### | | ####### | | ####### | |
| C | 21.1 | 21.6 | 21.2 | 21.2 | 20.4 | 20.5 | 20.7 | 21.1 | 20.0 | 20.6 | 20.6 | 21.1 |
| | ####### | | ####### | | ####### | | ####### | | ####### | | ####### | |
| D | 22.4 | 21.5 | 21.7 | 21.4 | 21.9 | 20.3 | 22.7 | 22.1 | 23.1 | 21.1 | 22.6 | 21.3 |
| | ####### | | ####### | | ####### | | ####### | | ####### | | ####### | |
| E | 24.7 | 21.6 | 24.1 | 20.7 | 24.1 | 21.3 | 24.3 | 19.8 | 24.0 | 21.1 | 25.7 | 20.9 |
| | ####### | | ####### | | ####### | | ####### | | ####### | | ####### | |
| F | 26.5 | 22.0 | 26.3 | 20.9 | 26.6 | 21.1 | 26.5 | 21.2 | 26.0 | 21.4 | 27.3 | 20.0 |
| | ####### | | ####### | | ####### | | ####### | | ####### | | ####### | |
| G | 20.2 | 21.3 | 20.1 | 21.0 | 20.0 | 20.6 | 20.2 | 20.2 | 20.6 | 21.2 | 19.9 | 20.3 |
| | ####### | | ####### | | ####### | | ####### | | ####### | | ####### | |
| H | 21.0 | 21.6 | 21.3 | 21.4 | 20.2 | 20.8 | | | 20.5 | 22.0 | 20.5 | 20.5 |
| | ####### | | ####### | | ####### | | ####### | | ####### | | ####### | |
| | 7 | | 8 | | 9 | | 10 | | 11 | | 12 | |
| | HCV | BVDV | HCV | BVDV | HCV | BVDV | HCV | BVDV | HCV | BVDV | HCV | BVDV |
| A | 19.5 | 20.5 | 19.9 | 19.9 | 20.1 | 20.4 | 20.3 | 20.0 | 28.4 | 20.4 | 45.0 | 24.1 |
| | ####### | | ####### | | ####### | | ####### | | ####### | | ####### | |
| B | 20.0 | 21.0 | 20.1 | 20.5 | 20.1 | 20.4 | 20.3 | 19.4 | 28.5 | 20.2 | 45.0 | 23.3 |
| | ####### | | ####### | | ####### | | ####### | | ####### | | ####### | |
| C | 20.6 | 20.7 | 20.2 | 19.6 | 20.5 | 21.2 | 21.1 | 20.2 | 23.6 | 20.8 | 28.8 | 24.1 |
| | ####### | | ####### | | ####### | | ####### | | ####### | | ####### | |
| D | 22.3 | 21.0 | 22.5 | 20.0 | 22.4 | 21.0 | 22.6 | 20.2 | 23.9 | 20.7 | 29.4 | 23.6 |
| | ####### | | ####### | | ####### | | ####### | | ####### | | ####### | |
| E | 25.4 | 21.2 | 25.4 | 20.0 | 25.4 | 21.1 | 25.2 | 19.5 | 20.9 | 20.7 | 23.2 | 24.0 |
| | ####### | | ####### | | ####### | | ####### | | ####### | | ####### | |
| F | 27.1 | 20.9 | 27.4 | 19.9 | 26.9 | 21.1 | 27.1 | 20.1 | 20.5 | 20.7 | 22.8 | 24.3 |
| | ####### | | ####### | | ####### | | ####### | | ####### | | ####### | |
| G | 19.7 | 21.0 | 19.9 | 20.8 | 19.9 | 20.6 | 20.1 | 19.9 | 45.0 | 20.5 | 16.0 | 22.5 |
| | ####### | | ####### | | ####### | | ####### | | ####### | | ####### | |
| H | 19.5 | 20.1 | 21.5 | 22.2 | | | 21.4 | 21.5 | 45.0 | 20.5 | 16.1 | 23.3 |
| | ####### | | ####### | | ####### | | ####### | | ####### | | ####### | |

1. A method of quantifying a first nucleic acid in a first biological source, comprising the steps of:
(a) combining said first biological source containing said first nucleic acid with a known amount of a second biological source containing a second nucleic acid;
(b) extracting from said combination said first nucleic acid and said second nucleic acid to form a combined nucleic acid extract;
(c) adding to said combined nucleic acid extract a first detectable probe which is specific for said first nucleic acid and a second detectable probe which is specific for said second nucleic acid;
(d) amplifying said combined nucleic acid extract by PCR means with a first set of primers which is specific for said first nucleic acid and a second set of primers which is specific for said second nucleic acid;
(e) quantifying at various PCR cycles during said amplification a detectable signal released independently from said first detectable probe and said second detectable probe;
(f) extrapolating the results of step (e) to calculate the amount of said first nucleic acid in said first biological source and the amount of said second nucleic acid in said second biological source; and
(g) evaluating accuracy of said calculated amount of said first nucleic acid determined in step (f) by comparing said calculated amount of said second nucleic acid in step (f) with said known amount of said second nucleic acid used in step (a).

2. The method according to claim 1 further comprising the step of adjusting said calculated amount of said first nucleic acid determined in step (f) of claim 1 by a factor determined by comparing said calculated amount of said second nucleic acid in step (f) of claim 1 with said known amount of said second nucleic acid used in step (a) of claim 1.

3. The method according to claim 1, wherein said first biological source is selected from cell-associated virus, including virus particles, subparticles, or free nucleic acid, and cell-free virus, including serum, plasma, or other media containing virus particles, subparticles, or free nucleic acid.

4. The method according to claim 1, wherein said first nucleic acid is selected from viral DNA or RNA from cell-associated or cell-free virus.

5. The method according to claim 1, wherein said second biological source is selected from cell-associated virus, including virus particles, subparticles, or free nucleic acid, and cell-free virus, including serum, plasma, or other media containing virus particles, subparticles, or free nucleic acid.

6. The method according to claim 1, wherein said amplification is conducted by PCR or RT-PCR.

7. The method according to claim 1, wherein said amplification is conducted using two sets of primers, wherein a first set of said primers is specific for said first nucleic acid and a second set of said primers is specific for said second nucleic acid.

8. The method according to claim 1, for quantifying nucleic acid in HCV, comprising the steps of:
(a) combining said HCV containing said first nucleic acid with a known amount of BVDV containing a second nucleic acid;
(b) extracting from said combination said first nucleic acid and said second nucleic acid to form a combined nucleic acid extract;
(c) adding to said combined nucleic acid extract with a first detectable probe which is specific for said first nucleic acid and a second detectable probe which is specific for said second nucleic acid;
(d) amplifying said combined nucleic acid extract by PCR or RT-PCR means;
(e) quantifying at various PCR cycles during said amplification a detectable signal released independently from said first detectable probe and said second detectable probe;
(f) extrapolating the results of step (e) to calculate the amount of said first nucleic acid in said HCV and the amount of said second nucleic acid in BVDV; and
(g) evaluating accuracy of said calculated amount of said first nucleic acid determined in step (f) by comparing said calculated amount of said second nucleic acid in step (f) with said known amount of said second nucleic acid used in step (a).

9. The method according to claim 8 further comprising the step of adjusting said calculated amount of said first nucleic acid determined in step (f) of claim 1 by a factor determined by comparing said calculated amount of said second nucleic acid in step (f) of claim 1 with said known amount of said second nucleic acid used in step (a) of claim 1.

10. A method of determining the effect of a compound on the replication of a first nucleic acid of a first biological source, comprising the steps of:
(a) combining said compound with a known amount of cell culture system to produce a first combination, wherein said first nucleic acid of said first biological source is capable of replication;
(b) after a time period combining said first combination with a second biological source containing a second nucleic acid to produce a second combination;
(c) extracting from said second combination said first nucleic acid and said second nucleic acid to form a combined nucleic acid extract;
(d) adding to said combined nucleic acid extract with a first detectable probe which is specific for said first nucleic acid and a second detectable probe which is specific for said second nucleic acid;
(e) amplifying said combined nucleic acid extract by PCR or RT-PCR means;
(f) quantifying at various PCR cycles during said amplification a detectable signal independently released from said first detectable probe and said second detectable probe;
(g) extrapolating the results of step (f) to calculate the amount of said first nucleic acid and said second nucleic acid in said second combination;
(h) evaluating accuracy of said calculated amount of said first nucleic acid determined in step (f) by comparing said calculated amount of said second nucleic acid in step (f) with said known amount of said second nucleic acid used in step (a);
(i) determining the effect of said compound on the replication of said first nucleic acid by comparing said amount of said first nucleic acid as determined in step (g) with the amount of said first nucleic nucleic acid determined separately in the absence of said compound.

11. The method according to claim 10, wherein said first biological source is selected from cell-associated hepatitis C virus, including virus particles, subparticles, or free nucleic acid, and cell-free hepatitis C virus, including serum, plasma, or other media containing virus particles, subparticles, or free nucleic acid

12. The method according to claim 10, wherein said compound is capable of inhibiting or interfering with Hepatitis C virus life cycle.

13. The method according to claim 10, wherein said second biological source is selected from cell-associated virus, including virus particles, subparticles, or free nucleic acid, and another cell-free virus, including serum, plasma, or other media containing virus particles, subparticles, or free nucleic acid.

14. The method according to claim 10, wherein said extraction means is selected from any suitable DNA or RNA extraction technique, including matrix-based single-well spin or vacuum column, or multiple-well extraction plate, or solution-based extraction methods

15. The method according to claim 10, wherein said first virus is HCV and said second virus is BVDV.

16. A method of simultaneously screening a plurality of compounds for their effect on the replication of a whole or part of a genome of a first biological source, comprising the steps of:
(a) placing in one or a plurality of wells said whole or part of a genome of said first biological and a medium suitable for replication of said genome;
(b) adding to each said well one or more of said compounds;
(c) adding to each said well a known amount of a second biological source as an internal control;
(d) using extraction means to extract together from each said well a first nucleic acid and a second nucleic acid to produce a combined nucleic acid extract from each well;
(e) amplifying and quantifying during the amplification process said first nucleic acid and said second nucleic acid in each well;
(f) determining the effect of each of said compounds on the replication of said whole or part of a genome of a first biological source using the results from step (e).
